(19) United States
(12) Patent Application Publication
(10) Pub. No.: US 2025/0109440 A1
(43) Pub. Date: Apr. 3, 2025

(54) DETECTION OF RARE CELLS IN A BLOOD SAMPLE

(71) Applicants: Roger Zemp, Edmonton (CA); Pradyumna Kedarisetti, Edmonton (CA)

(72) Inventors: Roger Zemp, Edmonton (CA); Pradyumna Kedarisetti, Edmonton (CA)

(21) Appl. No.: 18/376,379

(22) Filed: Oct. 3, 2023

(51) Int. Cl. C12Q 1/6886; C12Q 1/6806; C12Q 1/686

(52) U.S. Cl. CPC C12Q 1/6886; C12Q 1/6806; C12Q 1/686; C12Q 2600/158

(57) ABSTRACT

A method and apparatus for detecting rare cells in a blood sample. A test sample is obtained by enriching a concentration of nucleated cells in the blood sample. A treated sample is obtained by non-destructively treating the test sample to cause biomarkers to be released from the nucleated cells in the test sample. The measurement of one or more of a predetermined set of biomarkers indicative of rare cells in the treated sample is measured. The measurements from the treated sample are compared to baseline measurements obtained from an untreated sample to predict the presence of the rare cells in the blood sample.

### TECHNICAL FIELD

This relates to the detection of rare cells in a blood sample, and in particular, inducing the release of biomarkers from the rare cells.

### BACKGROUND

Metastatic disease is largely responsible for the majority of cancer related deaths. As such, the detection of cancer prior to metastatic spread can greatly improve patient outcomes as localized cancers may be excised surgically. However, early detection of metastasis is difficult to perform, as cancers are often not diagnosed until severe symptoms arise; this is partially due to the invasive and costly nature of current cancer diagnostics such as surgical biopsies which preclude rapid and continuous monitoring. These surgical approaches may also result in unintended recurrence and spread of cancer and their invasive nature may limit patient compliance.

Liquid biopsy approaches, such as blood and urine tests, offer a non-invasive diagnostic that can be performed rapidly and repeatedly with limited discomfort to patients. Liquid biopsies rely on the detection of biomarkers, often protein-based antigens, which are upregulated in certain disease states. Common markers are PSA for prostate cancer and CA125 for ovarian cancer. However, these often lack specificity to oncological disease as these proteins exist naturally with broadly varying levels and can often lead to over diagnosis as elevated PSA or CA125 levels may be caused by more common benign conditions such as prostatitis and uterine fibroids respectively. Over-diagnosis by these non-specific markers can lead to unneeded surgical treatments which negatively impacts patient comfort and can place a strain on medical systems.

In one example, with respect to prostate cancer, it is estimated that 48% of radical prostatectomies may have been unneeded. Several of these cases may have been due to elevated blood PSA levels due to prostatitis or abnormalities noted during digital rectal exams as these commonly employed methods that cannot reliably discriminate between slow growing, possibly benign cancer and cancers that may lead to metastatic disease. Furthermore, post-mortem studies of randomized men who died of non-cancer related causes found that 36%-51% of men died with prostate cancer rather than of prostate cancer. With the prevalence of easily accessible PSA tests, this data together suggests that not all cancer that is detected early may lead to metastatic cancer, and that early intervention in these cases may lead to unneeded patient discomfort and place an unneeded strain on the medical system.

More arduous and precise diagnostics, such as acquisition and analysis of surgical biopsies may be used to reduce overdiagnosis, however these procedures also come with a considerable level of patient discomfort and operator overhead.

### SUMMARY

According to an aspect, there is provided a method for detecting rare cells in a blood sample, the method comprising steps of: obtaining a test sample by enriching a concentration of nucleated cells in the blood sample; obtaining a treated sample by applying a non-destructive treatment to the test sample such that biomarkers are released from the nucleated cells in the test sample; measuring the concentration of one or more of a predetermined set of biomarkers indicative of rare cells in the treated sample; and comparing measurements from the treated sample to baseline measurements obtained from an untreated sample to predict the presence of the rare cells in the blood sample.

According to other aspects, the method may comprise one or more of the following features, alone or in combination: the baseline results may be obtained from the at least a portion of the test sample prior to treatment; the concentration of nucleated cells may be enriched using centrifugation, size separation, or magnetic separation; the rare cells may be circulating tumor cells (CTC), stem cells, activated T-cells, immune cells, or myeloma cells; the step of comparing measurements from a prior treated sample to determine a change in the presence of the rare cells in the blood samples over time; the predetermined set of biomarkers may be primarily present solely in the rare cells; the predetermined set of biomarkers may comprise miRNA; the predetermined set of biomarkers may comprises one or more of a group consisting of: hsa-miR-200c-3p200b-5p, hsa-miR-21200b-3p, hsa-miR-200c-3p, hsa-miR-200c, hsa-miR-125b-2-3p, hsa-miR-99a-3p, hsa-miR-23a-3p, hsa-miR-451a, hsa-miR-10b-5p, hsa-miR-21-5p, hsa-miR-182-5p, hsa-miR-602 and hsa-miR-767-5p; comparing the measurements to the baseline measurements may comprise measuring fold-change in miRNA panel levels, detecting one or more of the predetermined set of biomarkers may comprise using reverse transcription into cDNA, using quantitative Polymerase Chain Reaction (qPCR) with a thermocycler, using Loop-mediated Isothermal Amplification, using TaqMan™ advanced probes and primers, isolating miRNA using solid phase isolation, isolating miRNA using magnetic bead separation methods, isolating miRNA using liquid phase separation methods, identifying miRNA using gene-chip analysis, identifying miRNA using sequencing to identify miRNA, or combinations thereof; applying the non-destructive treatment comprises using ultrasonics, electromagnetics, thermal energy, chemicals, or combinations thereof; treating the at least a portion of the test sample may comprise injecting microbubbles or acoustically-active agents into the at least a portion of the test sample to enhance cell permeability; additional miRNA control sequences may be spiked into the test sample or the treated sample to normalize for differential loading, extraction efficiency, reverse transcription efficiency, and hemolysis; the rare cells may be treated such that a substantial portion of the rare cells in the treated sample are viable after treatment; the viable cells may be cultured after treatment; and the presence of the rare cells in the blood sample may be used to support a diagnosis of a disease.

According to an aspect, there is provided a system for detecting biomarkers in a blood sample, comprising a centrifuge or magnetic separation system for producing a test sample comprising an enriched concentration of nucleated cells in the blood sample; a non-destructive treatment device for treating the test sample to cause the nucleated cells to release biomarkers; and an analyzer for measuring released biomarkers in the test sample. The analyzer comprises a thermocycler or loop mediated isothermal amplifier for reverse transcription and qPCR and a fluorescence measurement sub-system. The system further comprises a processor for comparing a concentration of predetermined biomarkers in the test sample before and after treatment to generate a measure of rare cells in the test sample, the predetermined biomarkers being primarily expressed by the rare cells. The non-destructive treatment device may comprise an ultrasonic generator, an electromagnetic generator, a thermal energy generator, a microbubble injector, a chemical injector, or combinations thereof.

According to an aspect, there is provided a panel of one or more biomarkers indicative of circulating tumor cells in a blood sample, the panel being selected from the following miRNA: hsa-miR-200c-3p200b-5p, hsa-miR-21200b-3p, hsa-miR-200c-3p, hsa-miR-200c, hsa-miR-125b-2-3p, hsa-miR-99a-3p, hsa-miR-23a-3p, hsa-miR-451a, hsa-miR-10b-5p, hsa-miR-21-5p, hsa-miR-182-5p, hsa-miR-602, and hsa-miR-767-5p.

According to an aspect, there is provided a methodology for sensitive and specific detection of CTCs that may be used to preserve cell viability. The method may involve pre-enriching peripheral blood mononuclear cells, which may include white blood cells and CTCs, using simple density gradient centrifugation, followed by sonicating the cells using ultrasound, which may be done in the presence of microbubbles, to release biomarkers into the serum. A panel of micro-RNA (miRNA) markers that are specific to CTCs but largely absent in blood cells is then detected using reverse transcription and quantitative Polymerase Chain Reaction (qPCR). The sonication procedure creates transient pores in cells, releasing intracellular small molecules such as miRNAs. Our recent data with key prostate cancer cell lines and spike-in experiments with serial dilutions conclusively demonstrates single CTC detection from milliliters of whole blood.

Specific miRNA control sequences may be spiked into one or more processing steps to normalize for differential loading, extraction efficiency, and reverse transcription efficiency as well as hemolysis. Microbubbles or acoustically-active agents such as phase-change nanodroplets or nanobubbles to enhance sonoporation of cells may be used during the sonication procedure to enhance the porosity of the cells, or to enhance the release of biomarkers from the cells.

A calibration method may be used with the quantified miRNA biomarkers as a surrogate to detect and enumerate the rare cells.

There may be downstream processing of the rare cells which requires viable rare cells, the downstream processing comprising but not limited to cell culturing and drug testing

In other aspects, the features described above may be combined together in any reasonable combination as will be recognized by those skilled in the art.

### BRIEF DESCRIPTION OF THE DRAWINGS

These and other features will become more apparent from the following description in which reference is made to the appended drawings, the drawings are for the purposes of illustration only and are not intended to be in any way limiting, wherein:

FIG. 1 is a process overview of miRNA-based circulating tumor cells (CTC) diagnostics via high intensity focused ultrasound (HIFU) induced miRNA release.

FIG. 2 is a representative gene chip analysis of 20,000+ sequences to identify miRNA markers for CTCs that are largely absent in the background blood cells.

FIG. 3 are the test results of a single-CTC detection in blood with spike-in.

FIGS. 4A and 4B are a comparison of test results between samples with CTC and without CTC.

FIG. 5 are the test results of model CTCs in suspension normalized to zero HIFU treatment condition.

FIG. 6 is a summary of analysis showing linear scaling of miRNA signatures with prostatic cancer severity.

FIG. 7A-7E is a process overview of miRNA-based CTC diagnostics via HIFU induced miRNA release.

### DETAILED DESCRIPTION OF PREFERRED EMBODIMENTS

There will now be described a methodology that enriches a concentration of nucleated cells from a blood sample, identifies biomarkers, such as miRNA, present in rare cells of interest but that are absent in blood cells, and induces the rare cells to release the biomarkers such that they may be detected by analyzing the sample as a measure of the presence of the rare cells in the blood sample. This may involve, for example, refinement of miRNA isolation and cDNA synthesis, and may use qPCR data analysis with specialized controls for normalization. The methodology may be used to allow testing that is relatively simple and cost-effective and may be useful to triage for samples that contain rare cells, such as circulating tumor cells (CTCs), T-cells, stem cells, etc. relative to other methods, such as immunostaining validation and enumeration methods. If samples do contain the rare cells of interest, as validated with an ultrasound and qPCR method, subsequent tests may be undertaking such as gene expression analysis or gold-standard counting using FDA-approved methods such as CellSearch, ParSortix or Flow cytometry. If the rare cells are not present, then additional testing may be avoided. In the context of CTCs indicative of prostate cancer, this is important from a health-economics perspective as many men in a screening or active surveillance setting will not have clinically significant prostate cancer and many will have no CTCs in their blood. For those patients, costs may be reduced relative to other diagnostic tests.

CTCs may be used as a biomarker as they are primarily present in individuals with early to late-stage metastatic cancer, thereby suggesting much greater specificity than currently employed antigen tests. A hallmark of metastatic spread, CTCs are mobile daughter cells of a primary solid tumor that have been shed into circulation. During the formation of metastatic cancer, cancer cells invade from the primary site and intravasate into circulation, survive in circulation as circulating tumor cells, extravasate from circulation and attach and colonize at a distant metastatic site. If the presence of these mobile cancerous cells can be detected prior to metastatic spread, treatment can be planned accordingly, and the survival rates would improve substantially. As such, the early detection of cancer may have a profound effect on patient prognosis and level of treatment needed. The continual monitoring of metastatic spread may also greatly aid in determining the efficacy of treatment regiments.

The biomarkers that are released from the rare cells of interest may be small nucleic acids, such as miRNAs. Relative to protein biomarkers, small nucleic acids permit the use PCR amplification and the use of Taq-Man probes and primers, which are generally more sensitive and specific, and may be able to detect single copies, unlike protein assays. Advantages of miRNAs over mRNA or DNA is that miRNAs are smaller and can be released through sonoporation or related processes, which is less likely for larger nucleic acids. In order to achieve miRNA CTC detection from a blood sample, additional processing may be required as well as identification of a miRNA signature panel specific to CTCs with low basal expression in background blood cells. A panel may be defined for different types of rare cells to be detected, such as rare cells that are related to different types of cancers or other conditions that may result in rare cells being present in a blood sample. In addition to CTCs, examples of rare cells may include stem cells, activated T-cells, immune cells, or myeloma cells. In the discussion below, the biomarkers released from rare cells will be discussed in the context of miRNA released from CTC, with the understanding that similar principles may be applied to other rare cells that may release other suitable biomarkers.

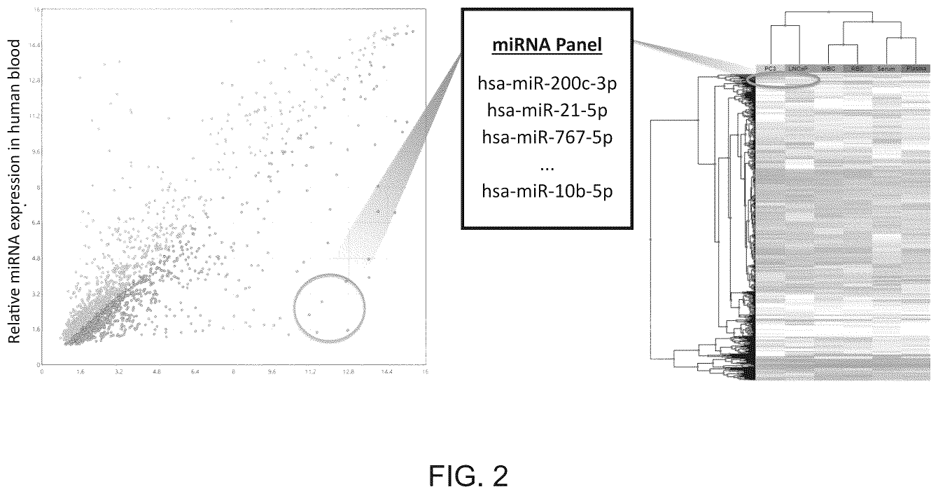

It has been found that spiked-in prostate cancer model CTCs may be detected with relatively high recovery rates and single-cell detection sensitivity using the discussed ultrasound biomarker release methods and qPCR. In these tests, LNCaP cells were spiked into whole human blood with various serial dilutions. As illustrated in FIG. 2, differential gradient centrifugation was used to isolate mononuclear cells and pellet out unwanted RBC populations.

In the detection approach discussed herein, high intensity focused ultrasound (HIFU) was used, referred to herein as sonication, to create short-lived pores in the cell membranes allowing miRNA to escape the cell and be detected in the surrounding fluid. The released microRNA serves as a surrogate marker for the presence of CTCs. miRNA are non-coding nucleic acids which are involved in gene regulation and are differentially released from varying cell types. By detecting miRNA signatures specific to CTCs of interest, the presence of CTCs (or other rare cells) in the blood sample may be measured.

While the examples and discussion below are given in terms of ultrasonics, other non-disruptive treatments may be applied to a test sample to cause biomarkers to be released from the cells in the test sample, either alone or in combination. For example, electromagnetics may be used to induce or encourage electroporation of cells. Thermal energy or chemical treatments may also be applied to encourage the cells to release biomarkers, and micro- or nanobubbles may be injected. The treatments may be designed, either alone or in combination with other types of treatments, to be applied in a non-disruptive process, such that the cells, or at least a majority of the cells, are not disrupted, and may remain viable, as the biomarkers are released. A process will be considered non-disruptive if a sufficient number of cells from which biomarkers are released remain to permit downstream processing. The treatment may also depend on the type of biomarkers being targeted. For example, a particular treatment may be more effective when targeting different types of biomarkers, such as miRNA, RNA, DNA, proteins, etc.

In some examples, when the non-disruptive treatment involves sonicating the sample, the sample may be sonicated with spiked-in microbubbles, which augment sonoporation effects on cells and facilitate biomarker release. Other treatments to enhance the porosity of the cells may include injecting lipofectamine, detergents, cationic lipids, nanoparticles, or nanobubbles, either alone or in combination. Biomarkers that are released include micro-RNAs, which are released more abundantly than larger molecules. The presence of rare cells may be detected by identifying a panel of a few miRNAs released from CTCs but that are absent in white blood cells. It was also found that, by enriching a concentration of nucleated cells in the blood sample, which primarily involves removing all or at least a substantial portion of red blood cells, the background “noise” associated with released biomarkers may be reduced. The blood sample may be separated using known techniques, and the test sample may be taken from the portion of the separated sample that contains the rare cells of interest.

To isolate the miRNA from the sample, beneficial results were obtained with solid phase microRNA adsorption using the QIAGEN microRNeasy kit. In this example, the supernatant samples are depleted of protein, lipid and large chain nucleic acid content and eluted into a stable buffer. The microRNA samples are then tagged with a poly-A tail and ligated to an adapter sequence which facilitates reverse transcription into cDNA.

The produced cDNA may then be quantized using qPCR. In one example, the detection method involved the use of the TaqMan™ system, which is generally recognized as having high sensitivity and specificity. The pre-sonication sample may then be compared to the post-sonication sample to look for a change in cycle threshold.

In one test, the fold-change in specific miRNA levels in plasma was plotted as a function of spiked in cells to demonstrate a several-fold-change when only one CTC was present, even when sonication was done in the presence of other white blood cells, which supports the conclusion that the approach used herein may offer single CTC detection performance in milliliters of blood. Moreover, it was found that the differential level of miRNAs detected scaled linearly with CTC count, which suggests that differential miRNA levels may be used as a surrogate for CTC enumeration.

Different levels of ultrasound were used that successfully preserved the viability of the cells in the sample, which is understood to mean about 80% or more of the cells in the sample.

The preservation of CTC viability may be useful for downstream analysis, to investigate the mechanisms of metastasis and determine anticancer treatment. Most notably, isolating viable CTCs may enable ex vivo culturing into larger amounts of cells allowing for multiomic analyses such as single-cell sequencing to obtain reliable data that reflect the characteristics of CTCs in the body. In a pre-clinical context, the isolation of viable CTCs may allow for the development of preclinical models and cell-lines for drug screening and modeling of the metastatic progression. Culturing of CTCs and testing in xenograft settings may also allow for the evaluation of anticancer therapeutic strategies prior to clinical implementation to inch closer towards realizing personalized medicine.

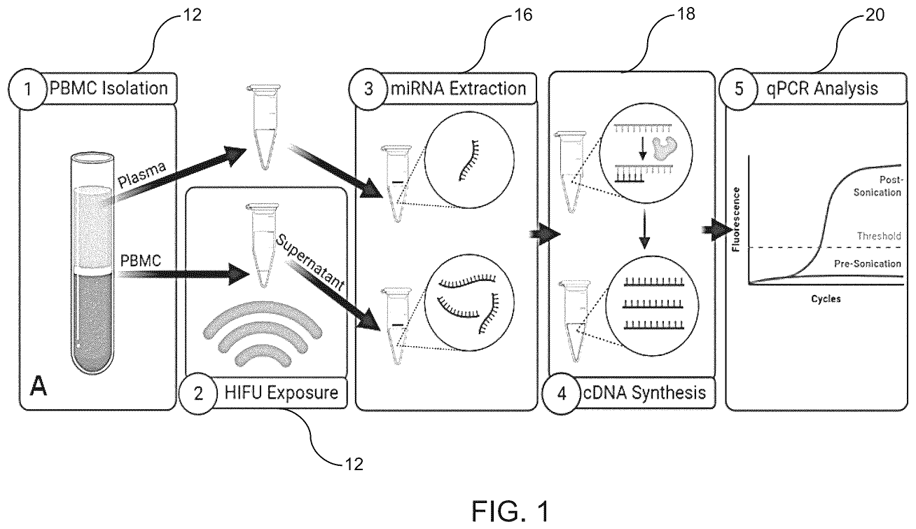

Referring to FIG. 1, a flow chart of a simplified process overview is shown. In step 12, peripheral blood mononuclear cells (PBMC) and plasma are isolated from a blood sample. In step 14, the PBMC sample is exposed to HIFU (of miRNA-based CTC diagnostics via HIFU to cause miRNA to be released in step 16. In step 18, cDNA synthesis is performed based on the released miRNA. In step 20, qPCR analysis is performed, and the results are analyzed for the presence of miRNA indicative of rare cells.

An example of the process is discussed in detail below.

### PBMC Isolation and High Intensity Focused Ultrasound Treatment

PBMC may be isolated from a blood sample using known techniques. In one example, whole blood samples of 5 mL from cancer naive patients were acquired prior to biopsy in K₂EDTA vacutainers. These samples were diluted 1:1 in 2% synthetic serum replacement in PBS and layer over 3.5 mL of differential gradient centrifugation media. These samples were centrifuged at 1200 g at 10° C. for 20 minutes. 6 mL of supernatant was decanted and the PBMC layer formed at the interface of the plasma and differential centrifugation media was isolated and further washed with 13 mL of 2% serum replacement in PBS and centrifuged for 10 min at 800 g at 10 C. The supernatant was decanted and discarded and the isolated PBMCs were resuspended in 0.75 mL of 2% serum replacement in PBS and placed in 1.5 mL DNA LoBind Tubes. These samples were then centrifuged for 5 minutes at 500 g at room temperature. 200 uL of the resulting supernatant was extracted to serve as the pre-sonication baseline. 150 uL of 2% serum replacement in PBS was added back into the cell solution along with 50 uL of the microbubble solution. The cell solution was resuspended by thorough but gentle pipetting and exposed to 80 m Vpp HIFU at 30% duty cycle for 5 minutes. After sonication, the cells were once again centrifuged at 500 g for 5 min at room temperature. 200 uL of the supernatant was extracted as the post-sonication sample. The precipitated PBMCs were frozen at −80 C for further analysis. In general, analytes are frozen at −80 C if storage is required for >12 hours.

### Microbubble Synthesis

Microbubbles or other techniques may be used to enhance sonoporation, when used. In one example, lipid-stabilized perfluorobutane-core microbubbles were created by first preparing a dry lipid film composed of 0.045 mg 1,2-dipalmitoyl-sn-glycero-3-phosphate (DPPA), 0.401 mg 1,2-dipalmitoyl-sn-glycero-3-phosphocholine (DPPC) and 0.304 mg 1,2-dipalmitoyl-sn-glycero-3-phosphoethanolamine-N-[methoxy (polyethylene glycol)-5000] (DPPE-mPEG5000) per milliliter of microbubble solution produced. Individual lipid components which were initially dissolved in chloroform were dispensed at the appropriate volumes into a round 5 mL bottom flask and the solvent was removed by evaporation on a rotavap operating at 474 mbar and 40° C. The lipid film was then hydrated with 1 mL of 1:1:8 glycerin:polypropylene glycol:PBS buffer. The lipid suspension was then loaded into 1.5 mL glass flasks with septum caps. The solution was heated to 80° C. and allowed to cool back to room temperature to redisperse lipid compounds. The air in the headspace was evacuated using a needle-syringe and replaced with ˜1.5 mL of perfluorobutane. The vial was then loaded into a VialMix and shaken thoroughly for 45 s to form microbubbles. The vial was then equilibrated to ambient pressure by releasing the pressure through the septum. After allowing to settle for 10 minutes at room temperature, the bottom half fraction of microbubbles were utilized in experiments to avoid the use of unstable large diameter microbubbles.

miRNA Isolation and cDNA Synthesis

After sonoporation, the released miRNA is isolated, and cDNA is synthesized. In one example, to improve the extraction of miRNA from the supernatant samples, a modified protocol was implemented using spin column-based nucleic acid purification kit (miRNeasy Serum/Plasma Advanced Kit, Qiagen). First, to control for variable sample loading, supernatant samples were spiked with 500 pg of cel-miR-2-3p. 200 uL of the sample was then aliquoted to a 1.5 mL tube and lysed using 200 uL RPL buffer followed by vortexing for 30 seconds and then left to further lyse over 10 minutes on a 1000 rpm shaker plate. The protein fractions in the samples were then precipitated by the addition of 60 uL RPP buffer, followed by vortexing for 30 seconds and left to further precipitate over 10 minutes on a 1000 rpm shaker plate. The precipitates were separated from the nucleic acid in solution by centrifugation at 12000 g for 5 minutes at room temperature. The supernatant was then isolated from the precipitate and the supernatant is mixed with one equivalent volume of isopropyl alcohol and thoroughly mixed. The sample was then loaded into the elution column and centrifuged for 30 seconds at 8000 g. The column is further washed with 700 uL 1:3 RWT buffer:isopropyl alcohol solution and centrifuged for 30 seconds at 8000 g. Another wash step was performed with 500 uL of 1:4 RPE buffer:ethanol solution and centrifuged for 30 seconds at 8000×g. A final wash was performed with 500 uL 80% isopropyl alcohol and centrifuged for 2 minutes at 12000×g. The column was then allowed to dry by centrifugation in vacuum centrifuge operating at 40° C. and 21000 g at 300 mbar pressure for 5 minutes. Once the columns were dry, the silica membrane was hydrated using 20 uL of pH 8.0 TE buffer and isolated miRNA was eluted by centrifugation at 21000 g for 1 minute.

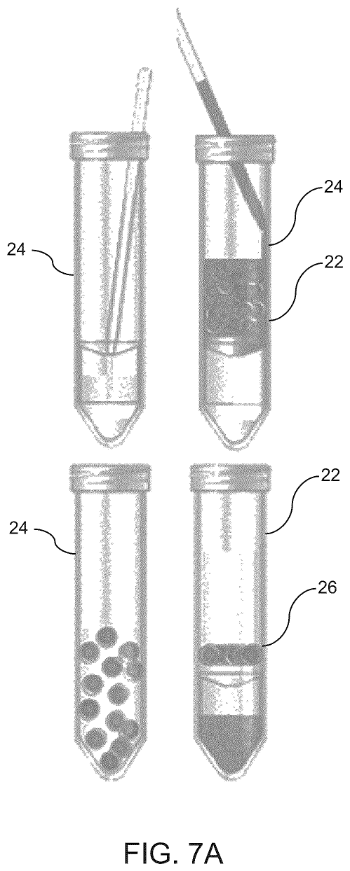

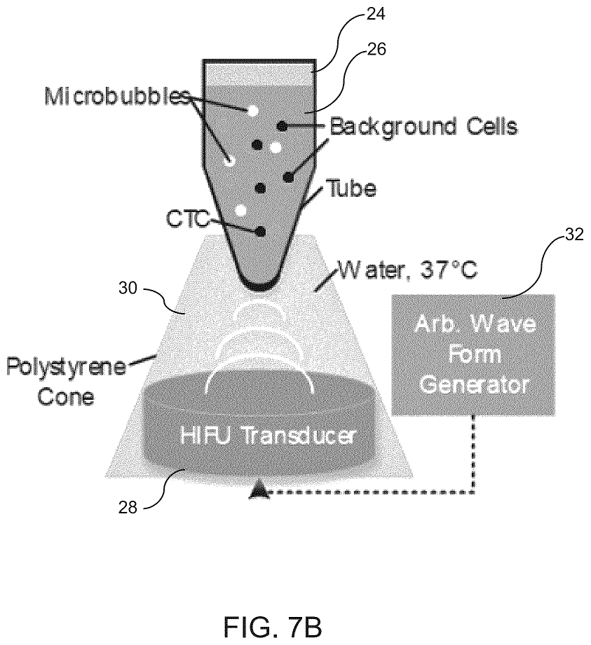

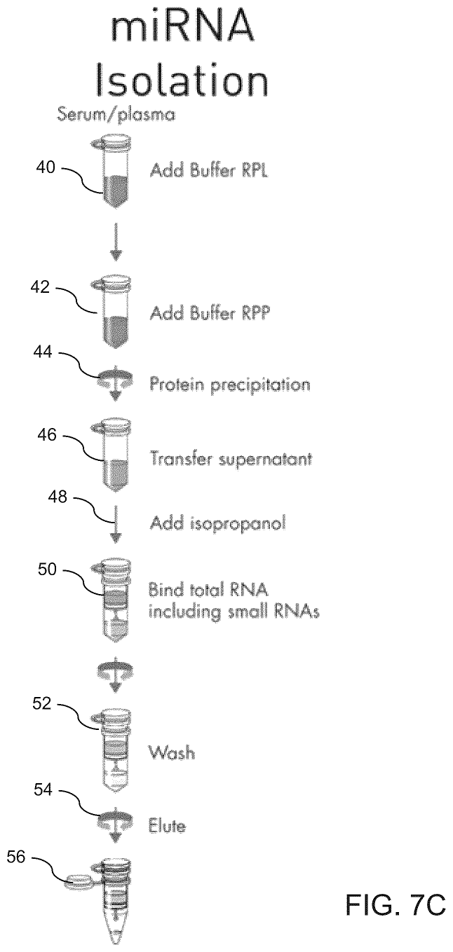

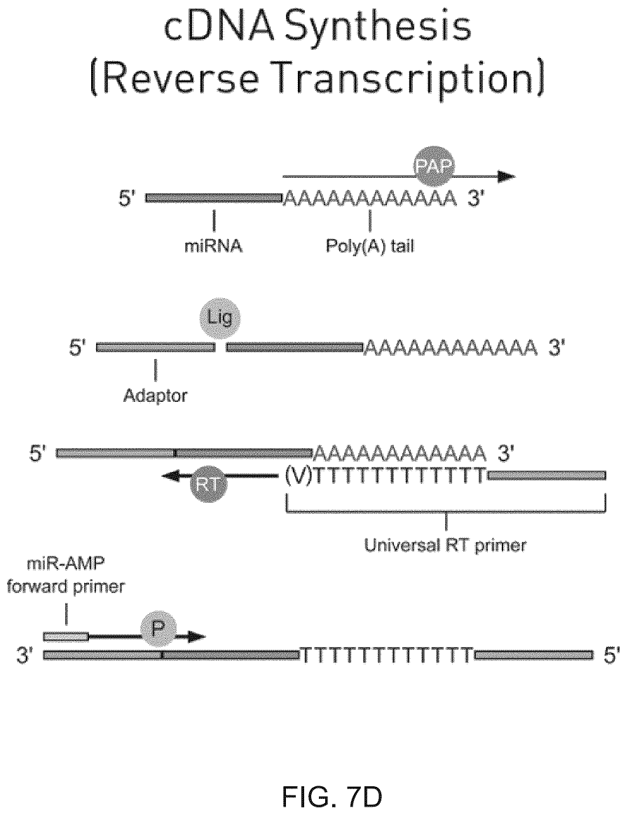

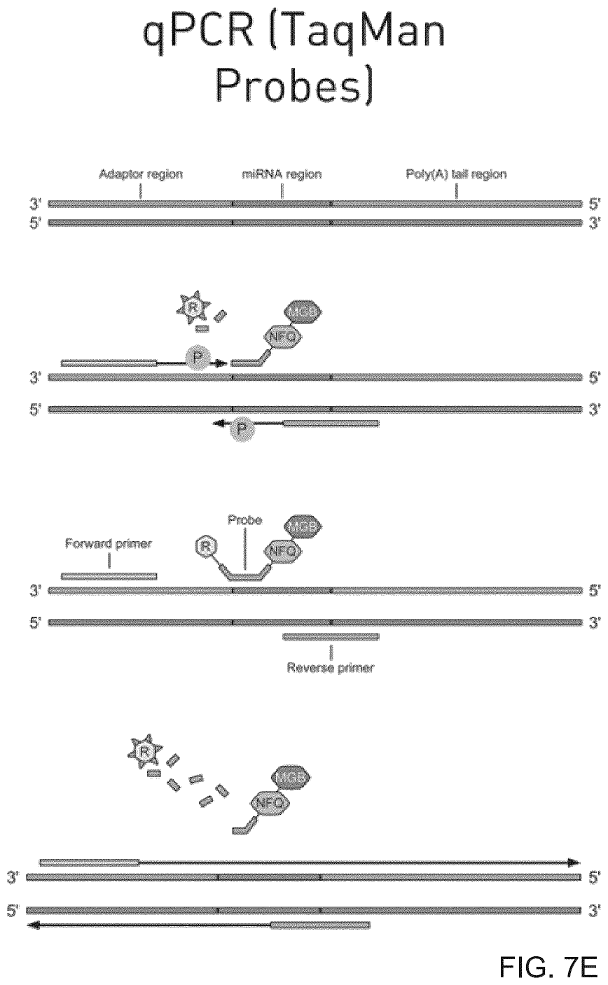

cDNA synthesis was performed using the TaqMan™ Advanced miRNA cDNA Synthesis Kit as per manufacturer recommended process. In short, a 3′ poly(A) tail was ligated to the sequence, followed by the addition of a 5′ adaptor sequence ligation and reverse transcription of the miRNA constructs was performed. An additional exogenous control sequence, cel-miR-39-3p was added at 500 pg per sample prior to ligation steps to control for differential reverse transcription efficiency between samples. Reverse transcription was then performed utilizing primers complementary to the adaptor sequence. Finally, a pre-amplification was performed utilizing primers complementary to 3′ poly (A) adaptor sequence for 15 cycles. An overview of this process flow is shown in FIG. 7, which is discussed in more detail below.

Selection of miRNA Panel

By selecting an appropriate panel of biomarkers that are present in rare cells but not white cells, the presence of rare cells may be detected. In one example, to identify miRNA candidates to discriminate between CTCs and background blood cells, model CTCs (LNCaP), healthy human blood fractions and patient human blood fractions were analyzed via GeneChip. Example results of this analysis are shown in FIG. 2, which shows a representative gene chip analysis of 20,000+ sequences. This allows putative miRNA markers to be identified for CTCs that are largely absent in the background blood cells. miRNA signatures that were overexpressed in PC3 and LNCaP cell lines and under-expressed in red blood cells, white blood cells and platelets were chosen as indicative of the rare cells to be detected.

miRNA Quantification

The treated sample was then analyzed to detect the presence of the expressed biomarkers, such as the miRNA in selected miRNA panel. In one example, quantitative real-time PCR was performed using FAM-MGB conjugated TaqMan™ Advanced miRNA Assays for the miRNA sequences listed in FIG. 2. For all cDNA samples, FAM fluorescence was detected during the annealing stage from 0 to 60 PCR cycles for all sequences run as singleplex reactions. Fluorescence detection curves were normalized to ROX passive reference dye included in the TaqMan™ Fast Advanced Master Mix and baselines and thresholds were maintained between assays for the same miRNA sequence and CT values for each product were determined at the center of log-linear region above the detection threshold. CT values for markers of interest were normalized to the expression levels of the exogenous control sequences, cel-miR-2-3p and cel-miR-39-3p, Ct*=Ct−Ct_{cel-miR-2-3p}−Ct_{cel-miR-39-3p}+Ct_{cel-miR-2-3p}+Ct_{cel-miR-39-3p} to normalize for differential miRNA extraction and reverse transcription efficiency. To control for the impact of hemolysis, the ratio of hsa-miR-451a and hsa-miR-23a-3p was monitored and ensured to be within 2 CT values between all samples. Each sample and assay combination were run in triplicate to ensure statistical rigor. For quality control, all qPCR reactions included additional non-reverse transcribed controls to identify genomic DNA contamination and a non-template control to identify reagent contamination. Final ΔCt values were determined as Ctₚₒₛₜ−Ctₚᵣₑ for each miRNA assay.

### Results

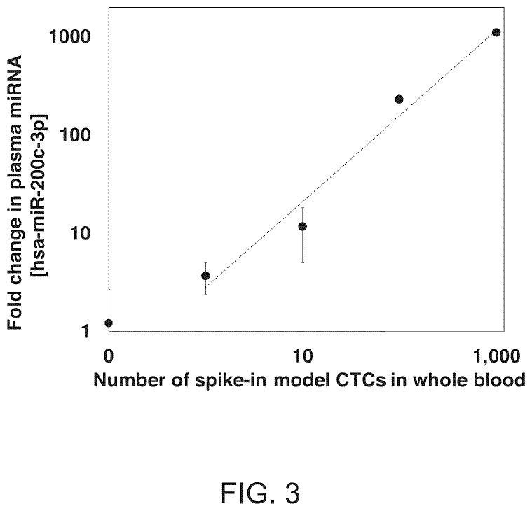

Quantizing the miRNA in the test samples allows the detection of the markers in the blood samples. In one example, to enable detection of these markers from lower blood sample volumes and with higher sensitivity, qPCR analysis was performed utilizing TaqMan™ probes and primers specific to the identified miRNA panel. Blood samples from various grades of prostate cancer were acquired pre-biopsy and processed using our approach. The results of spike-in experiments are shown in FIG. 3, demonstrating single-cell sensitivity of this approach and the experiments using patient blood samples to demonstrate the ability to distinguish between differing Gleason scores of prostatic cancer, as shown in FIG. 6.

One example of CTC detection within a blood sample without the need for arduous flow cytometry will now be discussed that shows that the CTC derived miRNA signature signal scales with prostatic cancer severity. Incorporation of this approach to standard PSA tests may enable improved distinction between clinically significant prostate cancer and benign conditions at a lower cost relative to some other CTC-based diagnostics such as CellSearch.

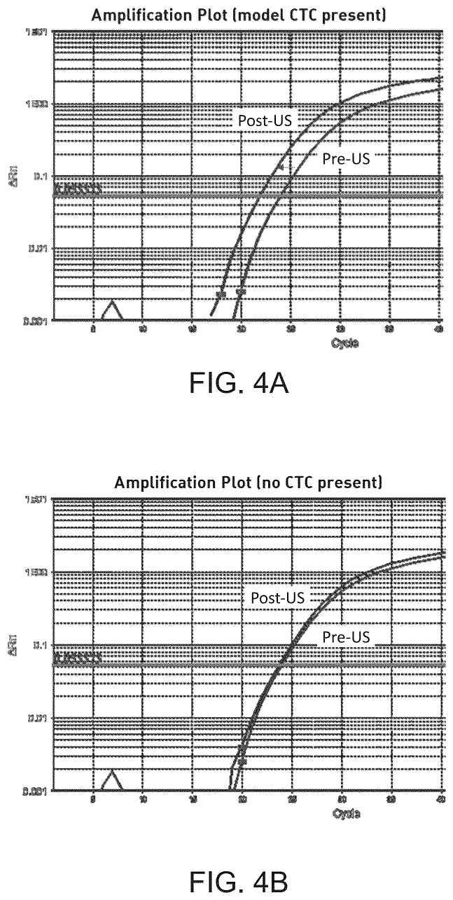

Referring to FIG. 4, representative qPCR curves are shown related to the proposed approach for CTC detection discussed herein. In samples containing CTCs, a decrease in Ct values was observed post-sonication but amplification of this signal was not observed if CTCs were not present. The change in signal is predicted to be proportional to the number of CTCs present in the sonicated sample.

### Cell Viability Assessment and Alternatives to Sonoporation

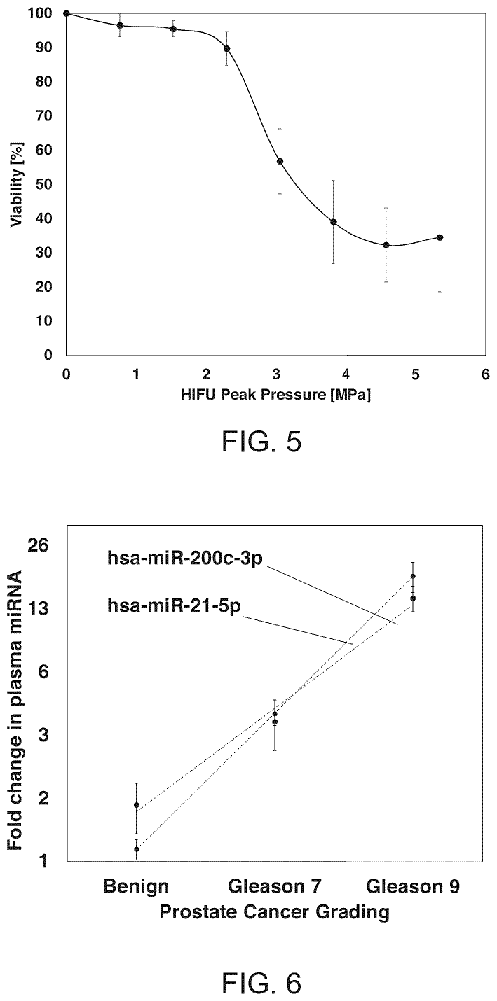

An example of cell viability as a function of sonication pressure is shown in FIG. 5, which represents the testing of model CTCs in suspension normalized to zero HIFU treatment condition, using a 50% duty cycle, and 5-minute exposure. As identified by MTT assay, sonication of cells in suspension at pressures below 3 MPa, the impact of treatment on cell viability is minimized. This allows for downstream culturing and analysis of identified CTCs enabling genomic studies and treatment response testing. Viable, in this context, refers to cells that, post membrane disruption, are able to repair and are still metabolically active and capable of undergoing mitosis, or are able to be cultured.

In detecting the presence of “rare cells”, the results may be indicative of conditions aside from cancer and may also be used to monitor the progress of disease, or the body's response to treatment. In particular, results over time may be compared to monitor the body's response to treatment, or detect changing conditions.

Referring to FIG. 6, the correlation of fold-change in miRNA with Gleason score/Grade Groups is depicted, demonstrating potential utility as a diagnostic tool. FIG. 6 is a summary of analysis showing linear scaling of miRNA signatures with prostatic cancer severity (n=3).

Referring to FIG. 7A-7E, a more detailed process overview of miRNA-based CTC diagnostics via HIFU induced miRNA release is depicted. Referring to FIG. 7A, a blood sample 22 is processed, such as in a test tube 24, to obtain a test sample 26 that has an enriched concentration of nucleated cells, such as PBMC.

Referring to FIG. 7B, microbubbles may be introduced into test sample 26 which may include CTC and background cells. Test tube 24 is subjected to sonication, such as by using a HIFU transducer 28, coupled to test tube 24 by water 30 or another medium and driven by an arbitrary waveform generator 32.

Referring to FIG. 7C, a flow chart of the procedure for miRNA isolation is shown. In step 40, RPL buffer is added; in step 42, RPP buffer is added. In step 44, protein is precipitated; in step 46, supernatant is transferred; in step 48, isopropanol is added; in step 50, the RNA, including small RNA, are bound; in step 52, the solution is washed and eluted in step 54. The RNA is isolated in step 56. In FIG. 7D, the process for cDNA synthesis is depicted. In FIG. 7E, the use of TaqMan™ probes in qPCR is shown.

In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context requires that there be one and only one of the elements.

The scope of the following claims should not be limited by the preferred embodiments set forth in the examples above and in the drawings but should be given the broadest interpretation consistent with the description as a whole.

What is claimed is:

1. A method for detecting rare cells in a blood sample, the method comprising steps of:
obtaining a test sample by enriching a concentration of nucleated cells in the blood sample;
obtaining a treated sample by applying a non-destructive treatment to the test sample such that biomarkers are released from the nucleated cells in the test sample and such that the nucleated cells are substantially undisrupted;
measuring one or more of a predetermined set of biomarkers indicative of rare cells in the treated sample; and
comparing measurements from the treated sample to baseline measurements obtained from an untreated sample to predict the presence of the rare cells in the blood sample.

2. The method of claim 1, wherein the baseline results are obtained from the at least a portion of the test sample prior to treatment.

3. The method of claim 1, wherein the concentration of nucleated cells is enriched using centrifugation, size separation, or magnetic separation.

4. The method of claim 1, wherein the rare cells are circulating tumor cells (CTC), stem cells, activated T-cells, immune cells, or myeloma cells.

5. The method of claim 1, further comprising the step of comparing measurements from a prior treated sample to determine a change in the presence of the rare cells in blood samples over time.

6. The method of claim 1, wherein the predetermined set of biomarkers are primarily present solely in the rare cells.

7. The method of claim 1, wherein the predetermined set of biomarkers comprises miRNA.

8. The method of claim 7, wherein the predetermined set of biomarkers comprises one or more of a group consisting of: hsa-miR-200c-3p200b-5p, hsa-miR-21200b-3p, hsa-miR-200c-3p, hsa-miR-200c, hsa-miR-125b-2-3p, hsa-miR-99a-3p, hsa-miR-23a-3p, hsa-miR-451a, hsa-miR-10b-5p, hsa-miR-21-5p, hsa-miR-182-5p, hsa-miR-602, and hsa-miR-767-5p.

9. The method of claim 7, wherein comparing the measurements to the baseline measurements comprises measuring fold-change in miRNA panel levels.

10. The method of claim 7, wherein detecting one or more of the predetermined set of biomarkers comprises using reverse transcription into cDNA; using quantitative Polymerase Chain Reaction (qPCR) with a thermocycler; using Loop-mediated Isothermal Amplification; using TaqMan™ advanced probes and primers; isolating miRNA using solid phase isolation; isolating miRNA using magnetic bead separation methods; isolating miRNA using liquid phase separation methods; identifying miRNA using gene-chip analysis; identifying miRNA using sequencing to identify miRNA; or combinations thereof.

11. The method of claim 1, wherein applying the non-disruptive treatment comprises using ultrasonic energy, electromagnetic energy, thermal energy, chemicals, or combinations thereof.

12. The method of claim 1, wherein applying the non-destructive treatment comprises sonicating the test sample and injecting microbubbles or acoustically-active agents into the test sample to enhance cell permeability.

13. The method of claim 1, wherein additional miRNA control sequences are spiked into the test sample or the treated sample to normalize for differential loading, extraction efficiency, reverse transcription efficiency, and hemolysis.

14. The method of claim 1, wherein a substantial portion of the rare cells in the treated sample are viable after treatment.

15. The method of claim 14, further comprising a step of culturing viable cells after treatment.

16. The method of claim 1, wherein the presence of the rare cells in the blood sample is used to support a diagnosis of a disease.

17. A system for detecting biomarkers in a blood sample, comprising:
a centrifuge or magnetic separation system for producing a test sample comprising an enriched concentration of nucleated cells in the blood sample;
a non-destructive treatment device for treating the test sample to cause the nucleated cells to release biomarkers;
an analyzer for measuring released biomarkers in the test sample, the analyzer comprising:
a thermocycler or loop mediated isothermal amplifier for reverse transcription and qPCR; and
a fluorescence measurement sub-system; and
a processor for comparing measurements of predetermined biomarkers in the test sample before and after treatment to generate a measure of rare cells in the test sample, the predetermined biomarkers being primarily released by the rare cells.

18. The system of claim 17, wherein the treatment device comprises an ultrasonic generator, an electromagnetic generator, a thermal energy generator, a microbubble injector, a chemical injector, or combinations thereof.

19. A panel of one or more biomarkers indicative of circulating tumor cells in a blood sample, the panel being selected from a group consisting of the following miRNA: hsa-miR-200c-3p200b-5p, hsa-miR-21200b-3p, hsa-miR-200c-3p, hsa-miR-200c, hsa-miR-125b-2-3p, hsa-miR-99a-3p, hsa-miR-23a-3p, hsa-miR-451a, hsa-miR-10b-5p, hsa-miR-21-5p, hsa-miR-182-5p, hsa-miR-602, and hsa-miR-767-5p.
